(19) United States
(12) Patent Application Publication
(10) Pub. No.: US 2015/0005207 A1
(43) Pub. Date: Jan. 1, 2015

(54) SYSTEMS AND METHODS FOR PRODUCING DIMETHYL SULFIDE FROM GASIFIED COKE

(71) Applicant: SHELL OIL COMPANY, Houston, TX (US)

(72) Inventors: Stanley Nemec MILAM, Houston, TX (US); Erik Willem TEGELAAR, Rijswijk (NL); John Justin FREEMAN, Pattison, TX (US); Richard B. TAYLOR, Sugar Land, TX (US)

(21) Appl. No.: 14/315,015

(22) Filed: Jun. 25, 2014

Related U.S. Application Data
(60) Provisional application No. 61/839,906, filed on Jun. 27, 2013.

(51) Int. Cl. C07C 319/14; C09K 8/58

(52) U.S. Cl. CPC C07C 319/14; C09K 8/58
USPC 507/257; 422/187; 568/60

(57) ABSTRACT

Petroleum coke or coal coke is gasified to produce a gas stream containing carbon monoxide, hydrogen, hydrogen sulfide, and optionally ammonia, carbon dioxide, water, and nitrogen. Carbon monoxide, hydrogen, and hydrogen sulfide, and optionally ammonia, carbon dioxide, water, and nitrogen are separated from the gas stream. The separated carbon monoxide and hydrogen are reacted to produce methanol, and the methanol is reacted with the separated hydrogen sulfide to produce dimethyl sulfide.

### RELATED CASES

This application claims benefit of U.S. Provisional Application No. 61/839,906, filed on Jun. 27, 2013, which is incorporated herein by reference.

### FIELD OF THE INVENTION

The present invention is directed to methods and systems of producing dimethyl sulfide from a gasified coke stream.

### BACKGROUND OF THE INVENTION

Coking is an important process in petroleum refining and coal processing. Relative to petroleum refining, coking is especially important in refining lower quality crudes that contain or, upon processing, form large amounts of high-boiling materials that are typically treated in coking units. Coking enables efficient conversion of these less desirable petroleum fractions to more desirable distillate products and a petroleum coke. Coal-coke is produced by a carbonization process of coal as a suitable reducing agent for iron oxide at elevated temperatures, and is produced, for example, as a reducing agent for iron ore in the production of steel from iron ore.

The coke (either petroleum coke or coal-coke) may then be gasified with steam and air to produce a gasified coke stream that includes carbon monoxide, carbon dioxide, hydrogen, nitrogen, and hydrogen sulfide. The relative ratios of the foregoing may depend on, inter alia, the composition of the coke being gasified and the coke gasification process. Typically the carbon dioxide and hydrogen sulfide are removed from the gasified coke stream leaving carbon monoxide, nitrogen and hydrogen which is used as fuel, for example to produce electricity. If steam and oxygen are used in the gasification process then nitrogen may be a minor component in the gasified coke stream.

However, many cokes have appreciable amounts of sulfur and would consequently yield gasified coke streams with high hydrogen sulfide concentrations. As the hydrogen sulfide is of little value and carries with it strict environmental regulations, high sulfur coke is typically not desirable. The concentration of the sulfur in the coke is related to the concentration of the sulfur in the petroleum or coal from which the coke is produced. Therefore, high sulfur petroleum and high sulfur coal are not viewed as valuable in the industry, and, therefore, are grossly under-produced.

Systems and methods that provide added value to high sulfur coke while addressing environmental concerns would be of value to those in the art.

### SUMMARY OF THE INVENTION

The present invention is directed to methods and systems of producing dimethyl sulfide from a gasified coke stream.

In one aspect, the present invention is directed to a method comprising:

providing a gasified coke stream comprising carbon monoxide, hydrogen, hydrogen sulfide, carbon dioxide, and nitrogen;

separating the gasified coke stream into a stream enriched in carbon monoxide relative to the gasified coke stream, a stream enriched in hydrogen relative to the gasified coke stream, and a stream enriched in hydrogen sulfide relative to the gasified coke stream;

producing methanol from at least a portion of the separated carbon monoxide enriched stream and at least a portion of the separated hydrogen enriched stream;

producing dimethyl sulfide from at least a portion of the produced methanol and at least a portion of the separated hydrogen sulfide enriched stream.

In another aspect, the present invention is directed to a system, comprising:

a separator that receives a gasified coke stream and is structured and arranged to produce a carbon monoxide stream, a hydrogen stream, and a hydrogen sulfide stream from the gasified coke stream;

a methanol reactor fluidly operatively coupled to the separator to receive at least a portion of the carbon monoxide stream and at least a portion of the hydrogen stream from the separator, wherein the methanol reactor is structured and arranged to produce a methanol stream from the carbon monoxide stream and the hydrogen stream; and

a dimethyl sulfide reactor fluidly operatively coupled to the methanol reactor to receive at least a portion of the methanol stream from the methanol reactor and fluidly operatively coupled to the separator to receive at least a portion of the hydrogen sulfide stream from the separator, wherein the dimethyl sulfide reactor is structured and arranged to produce a dimethyl sulfide stream from the methanol stream and the hydrogen sulfide stream.

The features and advantages of the present invention will be readily apparent to those skilled in the art upon a reading of the description of the preferred embodiments that follow.

### BRIEF DESCRIPTION OF THE DRAWINGS

The following figures are included to illustrate certain aspects of the present invention, and should not be viewed as exclusive embodiments. The subject matter disclosed is capable of considerable modifications, alterations, combinations, and equivalents in form and function, as will occur to those skilled in the art and having the benefit of this disclosure.

FIG. 1 is a diagram of a system in accordance with some embodiments of the present invention.

FIG. 2 is a diagram of a system in accordance with some embodiments of the present invention.

FIG. 3 is a diagram of a system in accordance with some embodiments of the present invention.

FIG. 4 is a graph showing petroleum recovery from oil sands at 30° C. using various solvents.

FIG. 5 is a graph showing petroleum recovery from oil sands at 10° C. using various solvents.

FIG. 6 is a graph showing the viscosity reducing effect of increasing concentrations of dimethyl sulfide on a West African Waxy crude oil.

FIG. 7 is a graph showing the viscosity reducing effect of increasing concentrations of dimethyl sulfide on a Middle Eastern Asphaltic crude oil.

FIG. 8 is a graph showing the viscosity reducing effect of increasing concentrations of dimethyl sulfide on a Canadian Asaphaltic crude oil.

### DETAILED DESCRIPTION

The present invention is directed to methods and systems of producing dimethyl sulfide from a gasified coke stream.

The systems and methods described herein utilize the hydrogen sulfide in a gasified coke stream to produce dimethyl sulfide, which can be particularly useful in enhanced oil recovery (“EOR”) methods. Further, such EOR methods typically utilize large quantities of dimethyl sulfide, which creates a larger market for high sulfur content coke, and consequently, high sulfur content petroleum and coal.

Further, because the systems and methods described herein react the hydrogen sulfide to form a relatively non-toxic product (i.e., dimethyl sulfide), many of the environmental issues previously associated with high sulfur coke can be mitigated.

As used herein, the term “coke” refers to both petroleum coke (i.e., coke derived from petroleum) and coal-coke (i.e., coke derived from coal).

As used herein, the term “high sulfur coke” refers to petroleum coke having about 3 wt. % or greater sulfur and coal-coke having about 1 wt. % or greater sulfur.

“Petroleum”, as used herein, is defined as a naturally occurring mixture of hydrocarbons, generally in a liquid state, which may also include compounds of sulfur, nitrogen, oxygen, and metals. As used herein, the term “petroleum” encompasses light hydrocarbons and heavy hydrocarbons. As used herein, the term “light petroleum” refers to petroleum having an API gravity of greater than 20°. As used herein, the term “heavy petroleum” refers to petroleum having an API gravity of at most 20°. Unless otherwise specified, as used herein the API gravity is determined in accordance with ASTM Method D4052.

“Fluidly operatively coupled or fluidly operatively connected,” as used herein, defines a connection between two or more elements in which the elements are directly or indirectly connected to allow direct or indirect fluid flow between the elements. The term “fluid flow”, as used in this definition, refers to the flow of a gas or a liquid; the term “direct fluid flow” as used in this definition means that the flow of a liquid or a gas between two defined elements flows directly between the two defined elements; and the term “indirect fluid flow” as used in this definition means that the flow of a liquid or a gas between two defined elements may be directed through one or more additional elements to change one or more aspects of the liquid or gas as the liquid or gas flows between the two defined elements. Aspects of a liquid or a gas that may be changed in indirect fluid flow include physical characteristics, such as the temperature or the pressure of a gas or a liquid; the state of the fluid between a liquid and a gas; and/or the composition of the gas or liquid. “Indirect fluid flow”, as defined herein, excludes changing the composition of the gas or liquid between the two defined elements by chemical reaction, for example, oxidation or reduction of one or more elements of the liquid or gas.

It should be noted that the terms “separate,” “separates,” “separating,” and the like, as used herein, do not necessarily imply a 100% degree of separation. Further, the term “stream” does not necessarily imply a purity level of the composition thereof.

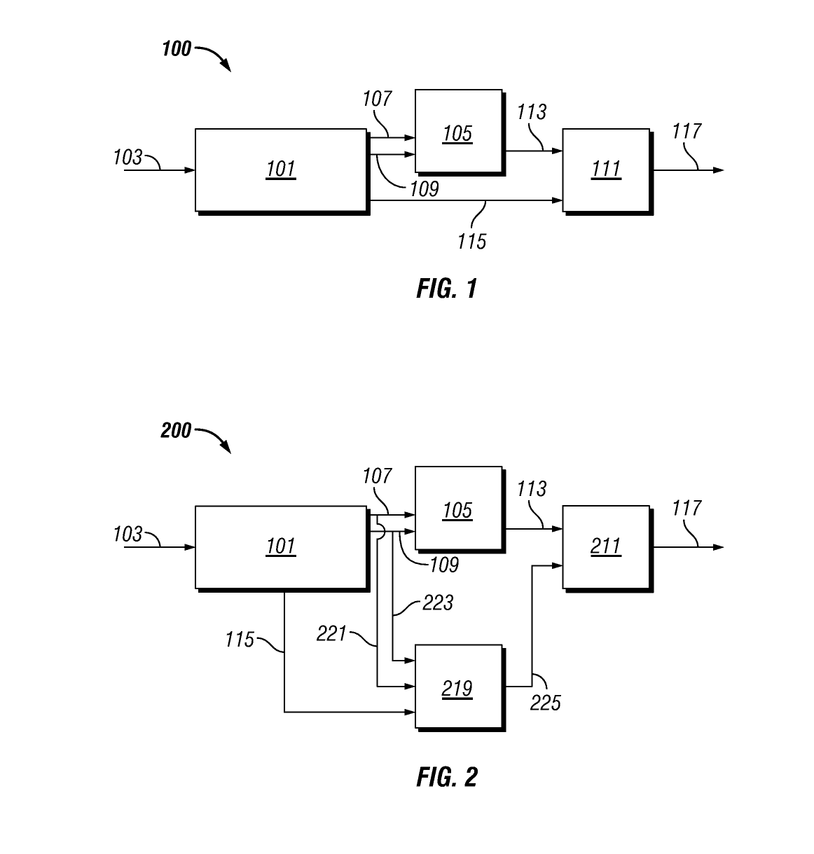

Systems 100 and 200 as shown in FIGS. 1 and 2, respectively, are systems in accordance with the present invention that may be utilized for conducting a process in accordance with the present invention. Each of the systems 100 and 200 of FIGS. 1 and 2, respectively, may be similar in some respects (e.g., similar system components or similar portions of the system may be understood with similar reference numerals). These should not be viewed as limiting. Other embodiments within the scope of the present invention would be evident to one of ordinary skill in the art.

In some embodiments, producing dimethyl sulfide from a gasified coke stream may include separating the carbon monoxide, hydrogen, and hydrogen sulfide from the gasified coke stream into individual streams enriched in carbon monoxide, hydrogen, and hydrogen sulfide, respectively; producing methanol from at least a portion of the carbon monoxide and at least a portion of the hydrogen; and producing dimethyl sulfide from at least a portion of the methanol and at least a portion of the hydrogen sulfide. Some embodiments may further involve producing the gasified coke stream by gasifying coke.

Coke suitable for use in conjunction with the methods and systems described herein may be petroleum coke, coal-coke, or any combination thereof. In some instances, petroleum coke may have a sulfur content of 0.5 wt. % or greater, or from 0.5 wt. % to 10 wt. %, or from 1 wt. % to 10 wt. %, or from 3 wt. % to 10 wt. %. In some instances, coal-coke may have a sulfur content of 0.5 wt. % or greater, or from 0.5 wt. % to 5 wt. %, or from 1 wt. % to 5 wt. %, or from 3 wt. % to 5 wt. %.

The coke may be gasified in a coke gasifier (not shown) to produce a gasified coke stream. Petroleum coke that is gasified may be produced in a delayed coker or a fluidized-bed coker. The coke gasifier may be any conventional coke gasifier used to gasify coke. The coke gasifier may be integrated with the coker and receive coke to be gasified as it is formed, or may not be integrated with coker and receive coke to be gasified from a storage facility.

Referring now to FIG. 1 illustrating an exemplary system of the present invention, the system 100 includes a separator 101 that receives a gasified coke stream via a conduit 103 and is structured and arranged to produce a carbon monoxide stream, a hydrogen stream, and a hydrogen sulfide stream from the gasified coke stream. A methanol reactor 105 is fluidly operatively coupled to the separator 101 to receive at least a portion of the carbon monoxide stream via a conduit 107 and at least a portion of the hydrogen stream via a conduit 109. The methanol reactor 105 is structured and arranged to produce a methanol stream from the carbon monoxide stream and the hydrogen stream. A dimethyl sulfide reactor 111 is fluidly operatively coupled to the methanol reactor 105 to receive at least a portion of the methanol stream via a conduit 113. The dimethyl sulfide reactor 111 is also fluidly operatively coupled to the separator 101 to receive a least a portion of the hydrogen sulfide stream via a conduit 115. The dimethyl sulfide reactor 111 is structured and arranged to produce a dimethyl sulfide stream, which may exit the dimethyl sulfide reactor 111 via a conduit 117. In some embodiments, not illustrated, system 100 may further include a coke gasification reactor that is structured and arranged to produce the gasified coke stream, wherein the separator 101 is fluidly operatively coupled to the coke gasification reactor to receive the gasified coke stream via the conduit 103.

As described above, gasified coke streams typically include carbon monoxide, hydrogen, hydrogen sulfide, carbon dioxide, and nitrogen. In some instance, the gasified coke stream may further include at least one of ammonia, water, and methane. The relative amounts of the foregoing may depend on, inter alia, the composition of the coke being gasified and the coke gasification process.

Separation of the gasified coke stream may be into streams of individual components, mixtures of individual components thereof, or both (e.g., a nitrogen stream, a nitrogen/hydrogen stream, or both). One of ordinary skill in the art, with the benefit of this disclosure, should recognize the plurality of methods and systems/apparatuses capable of separating gasified coke into the desired streams. For example, ammonia may be removed with a water absorber. Amine absorbers (e.g., N-methyl-diethanolamine) with high uptake capacity for hydrogen sulfide and low uptake capacity for carbon dioxide may be useful in selectively separating hydrogen sulfide from a gas stream. Refrigerated methanol (e.g., between about −74° C. (−100° F.) and −18° C. (0° F.)) or dimethyl ethers of polyethylene glycol may be useful in absorbing carbon dioxide, hydrogen sulfide (if present), and water. Cuprous aluminum chloride in an aromatic hydrocarbon solvent or cryogenic gas distillation processes may be useful in extracting carbon monoxide. Pressure swing gas absorption or cryogenic gas distillation processes may be useful in separating nitrogen from hydrogen.

In some embodiments, the separators 101 of the systems described herein may be structured and arranged to produce a carbon monoxide stream, a hydrogen stream, a hydrogen sulfide stream, and at least one of a carbon dioxide stream, a nitrogen stream, an ammonia stream, and combinations thereof.

One of ordinary skill in the art, with the benefit of this disclosure, should recognize the order of separation and additional cooling, compression, venting, return loops, storage tanks/facilities, and the like that can be included in a separator structured and arranged to produce the desired component streams or component mixture streams from a gasified coke stream.

Regarding the methanol reactor 105, one of ordinary skill in the art, with the benefit of this disclosure, should recognize conventional methods and systems/apparatuses capable of producing methanol from carbon monoxide and hydrogen. For example, methanol reactors may utilize catalysts having a mixture of copper, zinc oxide, and alumina at a pressure of about 5 MPa to about 10 MPa at a temperature of from 200° C. to 300° C. to produce methanol from carbon monoxide and hydrogen, often with high selectivity.

Regarding the dimethyl sulfide reactor 111, one of ordinary skill in the art, with the benefit of this disclosure, should recognize conventional methods and systems/apparatuses capable of producing dimethyl sulfide from methanol and hydrogen sulfide. For example, the dimethyl sulfide reactor may utilize a solid acid catalyst having moderate acidity such as a La2O3/Al2O3, γ-Al2O3, WO3/ZrO2, or WO3/Al2O3 catalyst for producing dimethyl sulfide from methanol and hydrogen sulfide. It should be noted that in the foregoing methods and systems, the production of dimethyl sulfide may advantageously include an excess of the stoichiometric amount of methanol used to produce dimethyl sulfide to minimize incomplete reaction that can yield significant quantities of methanethiol in the dimethyl sulfide product. Unlike dimethyl sulfide, methanethiol is reactive and toxic, and is preferably not produced in appreciable quantities in the methods described herein. In some instances, a recycle loop may be included to mitigate the production of methanethiol in appreciable quantities.

In some embodiments, producing dimethyl sulfide from a gasified coke stream may include separating the carbon monoxide, hydrogen, and hydrogen sulfide from the gasified coke stream into individual streams or separating hydrogen sulfide and a gas comprising the carbon monoxide and hydrogen into separate streams; producing methanol from a portion of the separated carbon monoxide and the separated hydrogen streams or from the separated gas comprising the carbon monoxide and hydrogen; producing methanethiol from a portion of the carbon monoxide stream, the hydrogen sulfide stream, and optionally a portion of the hydrogen stream, or from a portion of the gas comprising the carbon monoxide and hydrogen and the hydrogen sulfide stream; and producing dimethyl sulfide from the methanol and the methanethiol. Some embodiments may further involve producing the gasified coke stream by gasifying coke.

Referring now to FIG. 2 illustrating an exemplary system of the present invention, the system 200 includes a separator 101 that receives a gasified coke stream via a conduit 103 and is structured and arranged to produce a carbon monoxide stream, a hydrogen stream, and a hydrogen sulfide stream from the gasified coke stream. A methanol reactor 105 is fluidly operatively coupled to the separator 101 to receive a portion of the carbon monoxide stream via a conduit 107 and a portion of the hydrogen stream via a conduit 109. The methanol reactor 105 is structured and arranged to produce a methanol stream from the carbon monoxide stream and the hydrogen stream received therein. System 200 also includes a methanethiol reactor 219 that is fluidly operatively coupled to the separator 101 to receive a portion of the carbon monoxide stream via a conduit 221, a portion of the hydrogen stream via a conduit 223, and at least a portion of the hydrogen sulfide stream via a conduit 115. The methanethiol reactor 219 is structured and arranged to produce a methanethiol stream from the carbon monoxide stream, the hydrogen stream, and the hydrogen sulfide stream received therein. A dimethyl sulfide reactor 211 to is fluidly operatively coupled to the methanol reactor 105 to receive the methanol stream via a conduit 113 and to the methanethiol reactor 219 to receive the methanethiol stream via a conduit 225. The dimethyl sulfide reactor 211 is structured and arranged to produce a dimethyl sulfide stream from the methanol stream and the methanethiol stream received therein. The dimethyl sulfide stream may exit the dimethyl sulfide reactor 211 via a conduit 117. In some embodiments, not illustrated, system 200 may further include a coke gasification reactor that is structured and arranged to produce the gasified coke stream, wherein the separator 101 is fluidly operatively coupled to the coke gasification reactor to receive the gasified coke stream via the conduit 103.

Regarding the methanethiol reactor 219, one of ordinary skill in the art, with the benefit of this disclosure, should recognize conventional methods and systems/apparatuses capable of producing methanethiol from the carbon monoxide, hydrogen and the hydrogen sulfide. For example, a methanethiol reactor may utilize catalyst systems that include K2MoO4 for producing methanethiol from carbon monoxide, hydrogen and hydrogen sulfide.

Regarding the dimethyl sulfide reactor 211, one of ordinary skill in the art, with the benefit of this disclosure, should recognize conventional methods and systems/apparatuses capable of producing dimethyl sulfide from methanol and methanethiol. For example, the dimethyl sulfide reactor may utilize solid acid catalyst systems having moderate acidity, e.g. La2O3/Al2O3, γ-Al2O3, WO3/ZrO2, or WO3/Al2O3 catalysts, for producing dimethyl sulfide from methanol and methanethiol. It should be noted that in the foregoing methods and systems, the production of dimethyl sulfide may advantageously include an excess of the stoichiometric amount of methanol used to produce dimethyl sulfide to minimize unreacted methanethiol in the dimethyl sulfide product.

The dimethyl sulfide produced in the methods and systems described herein may, in some embodiments, be used in making an oil recovery formulation. In some embodiments, the oil recovery formulation may comprise at least 75 mol % dimethyl sulfide. In some instances, the oil recovery formulation may comprise at least 80 mol %, or at least 85 mol %, or at least 90 mol %, or at least 95 mol %, or at least 97 mol %, or at least 99 mol % dimethyl sulfide. In some instances, the oil recovery formulation may consist essentially of dimethyl sulfide, or may consist of dimethyl sulfide.

In some instances, the oil recovery formulation may comprise dimethyl sulfide and one or more co-solvents. The one or more co-solvents are preferably miscible with dimethyl sulfide. Examples of suitable co-solvents may include, but are not limited to, o-xylene, toluene, carbon disulfide, dichloromethane, trichloromethane, C3-C8 aliphatic and aromatic hydrocarbons, natural gas condensates, hydrogen sulfide, diesel, kerosene, dimethyl ether, decant oil, and mixtures thereof. In some embodiments, water is absent from the oil recovery formulation (i.e., no additional water than residual water concentrations in the components of the oil recovery formulation under ambient conditions).

In some instances, the oil recovery formulation described herein preferably is relatively non-toxic or is non-toxic. The oil recovery formulation may have an aquatic toxicity of LC50 (rainbow trout) greater than 200 mg/1 at 96 hours. The oil recovery formulation may have an acute oral toxicity of LD50 (mouse and rat) of from 535 mg/kg to 3700 mg/kg, an acute dermal toxicity of LD50 (rabbit) of greater 5000 mg/kg, and an acute inhalation toxicity of LC50 (rat) of at least 40250 ppm at 4 hours.

In some instances, the oil recovery formulation described herein preferably has a relatively low density (e.g., at most 0.9 g/cm3, or at most 0.85 g/cm3).

In some instances, the oil recovery formulation described herein may have a relatively high cohesive energy density (e.g., from 300 Pa to 410 Pa, or from 320 Pa to 400 Pa).

In some embodiments, the amount of methanol produced by the methods and systems described herein may be in excess of what is needed for producing the desired amount of dimethyl sulfide. As such, a portion of the methanol produced may be stored, transported to another location, directed to other reactors, or any combination thereof. Examples of other reactors may include, but are not limited to, methanol-to-gasoline reactors for producing gasoline, methanol-to-olefin reactors for producing olefins, and dimethyl ether reactors for producing dimethyl ether. Therefore, in some embodiments, systems described herein may further comprise a methanol-to-gasoline reactor fluidly operatively coupled to the methanol reactor to receive a portion of the methanol stream via a conduit and structured and arranged to produce a gasoline stream. In some embodiments, systems described herein may further comprise a methanol-to-olefin reactor fluidly operatively coupled to the methanol reactor to receive a portion of the methanol stream via a conduit and structured and arranged to produce an olefin stream. In some embodiments, systems described herein may further comprise a dimethyl ether reactor fluidly operatively coupled to the methanol reactor to receive a portion of the methanol stream via a conduit and structured and arranged to produce a dimethyl ether stream.

Further, in some embodiments, the components or component mixtures from the gasified coke stream may be in excess of what is needed for producing the desired amount of dimethyl sulfide. As such, the components or component mixtures from the gasified coke stream may be each independently stored, transported to another location, directed to other reactors, or a combination thereof. For example, carbon dioxide may be compressed, liquefied and utilized in EOR methods. In some embodiments, the system described herein may further comprises a compressor, condenser, and storage facility fluidly operatively coupled to the separator to receive the compressed, condensed carbon dioxide stream via a conduit.

In some instances, the amount of at least one of the components or component mixtures from the gasified coke stream may be insufficient to produce the desired amount of dimethyl sulfide. As such, the systems and methods described herein may optionally include additional input streams for the components or component mixtures from the gasified coke stream. For example, the hydrogen sulfide from sour gas may be an additional input stream for the methods or systems described herein. In another example, the carbon monoxide and hydrogen produced from a methane reactor (e.g., an autothermal reformer, a steam methane reformer, a catalytic partial oxidation reactor, a partial oxidation reactor, or the like) may be an input for the methods or systems described herein.

In some embodiments, the systems described herein may further include a sour gas separator that is structured and arranged to produce a methane stream and a hydrogen sulfide stream. In some instances, the hydrogen sulfide stream from the coke gas separator 101 may be combined with a hydrogen sulfide stream from the sour gas separator and the combined hydrogen sulfide stream may be utilized to produce dimethyl sulfide in the dimethyl sulfide reactor 111 or to produce methanethiol in the methanthiol reactor 219. In some instances, the dimethyl sulfide reactor 111 or the methanethiol reactor 219 may be fluidly operatively coupled to the sour gas separator to receive the hydrogen sulfide stream therefrom.

In some embodiments, the systems described herein may further comprise a methane reactor that is structured and arranged to produce a carbon monoxide stream and a hydrogen stream from a methane stream received therein (from the sour gas separator or otherwise). The methane reactor may be an autothermal reformer, a steam-methane reformer, a catalytic partial oxidation reactor, or a partial oxidation reactor. The methane reactor may be fluidly operatively coupled to the methanol reactor, or to the methanethiol reactor, or to both to provide the carbon monoxide stream and the hydrogen stream produced by the methane reactor to the methanol reactor or to the methanethiol reactor.

While compositions and methods are described in terms of “comprising” various components or steps, the compositions and methods can also “consist essentially of” or “consist of” the various components and steps. When “comprising” is used in a claim, it is open-ended.

All numbers expressing quantities of ingredients, properties such as molecular weight, reaction conditions, and so forth used in the present specification and associated claims may be understood as being modified by the term “about.”

To facilitate a better understanding of the present invention, the following examples of preferred or representative embodiments are given. In no way should the following examples be read to limit, or to define, the scope of the invention.

### EXAMPLES

### Illustrative Example

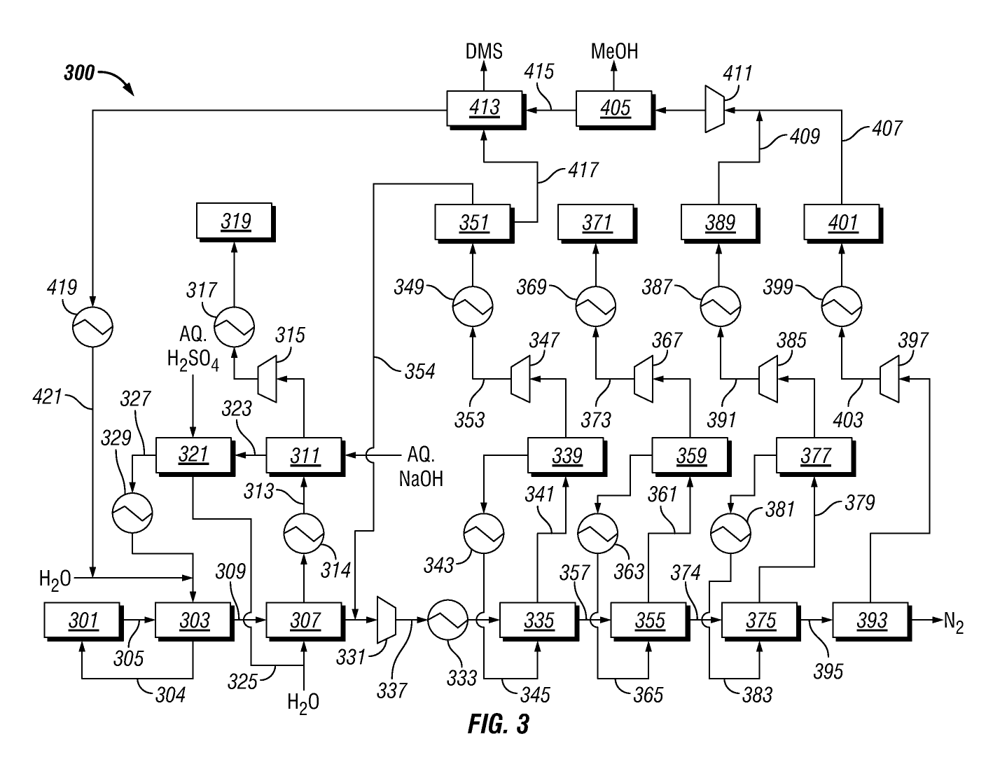

Referring now to FIG. 3, a system 300 for conducting a process in accordance with the present invention is shown. A gas stream containing carbon monoxide, hydrogen, hydrogen sulfide, and optionally ammonia, nitrogen, carbon dioxide, and water and having a temperature of from 830° C. to 1000° C. may be produced in a coker 301, for example a flexicoker or fluidized-bed coker with an integrated gasifier. The coker 301 may be fluidly operatively coupled to a boiler 303 via conduit 305 to provide the gas stream to the boiler 303. The boiler 303 may be structured and arranged to effect heat exchange between the gas stream and a water stream to cool the gas stream to a temperature of from 25° C. to 80° C. and to heat the water stream to produce steam. The steam produced in the boiler 303 may be used to provide thermal energy to the coker, for example, if the coker is a flexicoker, steam may be provided from the boiler 303 to the coker 301 via conduit 304, or the steam may be used to provide thermal energy to other portions of the system requiring thermal energy. The cooled gas stream may be provided from the boiler 303 to an ammonia absorber 307 that is fluidly operatively coupled to the boiler via conduit 309. The ammonia absorber 307 may be structured and arranged to contact the cooled gas stream with water at a temperature of from 5° C. to 60° C. to wash substantially all of the ammonia and a portion of the carbon dioxide and a portion of the hydrogen sulfide from the cooled gas stream to produce an ammonia-depleted gas stream and an ammonia-enriched water stream.

Ammonia may be separated and recovered from the ammonia-rich water stream to regenerate the water for re-use in the ammonia absorber 307. The ammonia absorber 307 may be fluidly operatively coupled to an ammonia stripper 311 by conduit 313, and the ammonia-enriched water stream may be provided from the ammonia absorber to the ammonia stripper. The pH of the ammonia-enriched water stream may be adjusted to a pH of 10-12 by adding aqueous sodium hydroxide to the ammonia-enriched water stream. The ammonia-enriched water stream may be heated in a heat exchanger 314 to a temperature of from 65° C. to 90° C. prior to entering the ammonia stripper or may be heated upon entering the ammonia stripper to release ammonia gas from the ammonia-enriched water stream. The released ammonia gas may be separated from the ammonia-enriched water stream in the ammonia stripper 311, then compressed in an ammonia compressor 315 to pressure of 0.5 MPa to 3.1 MPa and cooled in a heat exchanger 317 to a temperature of from 10° C. to 50° C. to produce liquid ammonia, which may be stored in a liquid ammonia storage tank 319. The ammonia stripper 311 may be fluidly operatively coupled to the liquid ammonia storage tank 319 to provide ammonia thereto. The pH of the ammonia-depleted water steam from ammonia stripper 311 may be adjusted to a pH of from 5-7 with an aqueous acid such as aqueous H2SO4 in an acid-gas stripper 321 that is fluidly operatively coupled to the ammonia stripper 311 by conduit 323. Hydrogen sulfide and carbon dioxide gases may be stripped from the ammonia-depleted water stream in the acid-gas stripper 321 at a temperature of from 65° C. to 120° C. and recombined with the ammonia-depleted gas stream via conduit 325. The ammonia, hydrogen sulfide, carbon dioxide stripped water stream may be cooled in heat exchanger 329 and recycled to the boiler 303 via conduit 327.

The ammonia-depleted gas stream may be treated to separate H2S preferentially from CO2, water, CO, N2, and H2. The ammonia-depleted gas stream may be compressed to a pressure of 2.1 MPa to 6.9 MPa in a compressor 331 and the compressed ammonia-depleted gas stream may be cooled against cooling water in a heat exchanger 333 to a temperature of 10° C. to 50° C. and provided to an H2S absorber 335, where the H2S absorber may be fluidly operatively coupled to the ammonia absorber 307 via conduit 337. The H2S absorber 335 may be a scrubber in which the compressed ammonia-depleted gas stream may be contacted at a temperature of 10° C. to 50° C. with a solvent having a high uptake capacity for H2S gas but a very low uptake capacity for CO2, CO, H2, and N2 gases. Preferred solvents for selectively absorbing H2S include N-methyl-diethanol amine (MDEA), aqueous MDEA, N-methyl-2-pyrrolidone (NMP), and aqueous NMP. A H2S-depleted gas stream and an H2S-enriched solvent stream may be produced by contacting the ammonia-depleted gas stream with the H2S selective solvent in the H2S absorber 335.

The H2S-enriched solvent stream may be treated to separate H2S and any CO2 therein from the solvent. The H2S absorber 335 may be fluidly operatively coupled to an H2S solvent regenerator 339 to provide the H2S-enriched solvent to the H2S solvent regenerator via conduit 341. The H2S enriched solvent may be heated to a temperature of from 115° C. to 135° C. at a pressure of from 0.101 MPa to 0.17 MPa in the H2S solvent regenerator 339 to regenerate the solvent by off-gassing the H2S and any CO2 from the H2S-enriched solvent. The regenerated solvent may be cooled against cooling water in heat exchanger 343 to a temperature of from 10° C. to 50° C. and returned to the H2S absorber 335, where the H2S absorber may be fluidly operatively coupled to the H2S solvent regenerator to receive the regenerated solvent via conduit 345. The off-gassed stream of H2S and CO2 may be compressed in a compressor 347 to a pressure of from 0.7 MPa to 6.9 MPa and then cooled against cooling water in heat exchanger 349 to a temperature of from 10° C. to 65° C. to liquefy the H2S. The liquefied H2S may be dried and stored in H2S storage tank 351, which may be fluidly operatively couped to the H2S solvent regenerator 339 via conduit 353. The liquid H2S may be stored at a pressure below 3.4 MPa but sufficient to maintain H2S in the liquid phase so that any CO2 gas present in the liquefied H2S may be purged from the liquid H2S. The purged CO2 gas may be recombined with the ammonia-depleted gas stream via conduit 354 for compression in compressor 331 and reintroduction into the H2S absorber 335.

The H2S-depleted gas stream may be treated to separate CO2, water, and any residual H2S from CO, N2, and H2. The H2S-depleted gas stream may be provided to a CO2 absorber 355, where the CO2 absorber may be fluidly operatively coupled to the H2S absorber 335 via conduit 357 to receive the H2S-depleted gas stream. The CO2 absorber 355 may be a high capacity scrubber in which the H2S-depleted gas stream may be contacted with a solvent having a high uptake capacity for CO2 and high selectivity for CO2 uptake relative to CO, H2, and N2 in the H2S-depleted gas stream. The solvent for use in the CO2 absorber is preferably methanol or methanol/water containing at least 50 wt. % methanol, wherein the CO2 selective solvent has a temperature of from −20° C. to −75° C. A CO2-depleted gas stream and a CO2-enriched solvent stream may be produced by contacting the H2S-depleted gas stream with the CO2 selective solvent in the CO2 absorber 355.

The CO2-enriched solvent stream may be treated to separate CO2 from the solvent. The CO2 absorber 355 may be fluidly operatively coupled to a CO2 solvent regenerator 359 to provide the CO2-enriched solvent to the CO2 solvent regenerator via conduit 361. The CO2 selective solvent may be regenerated and a CO2 off-gas stream may be produced by decompressing the CO2-enriched solvent stream to a pressure at which CO2 is degassed from the CO2 selective solvent based on a standard CO2 saturated vapor pressure curve—for example to a pressure of less than 1.0 MPa at −40° C., or a to a pressure of less than 1.5 MPa at −28.9° C., or to a pressure of less than 2.1 MPa at −17.8° C.—or may be heated to a temperature at which CO2 is degassed from the CO2 selective solvent—for example to a temperature of from 0° C. to 35° C. at a pressure of less than 2.5 MPa—or both. A selected amount of water may be separated from the regenerated CO2 selective solvent by distillation if desired, and the regenerated CO2 selective solvent may be cooled in heat exchanger 363 and returned to the CO2 absorber 355, where the CO2 absorber may be fluidly operatively coupled to the CO2 solvent regenerator 359 to receive the regenerated CO2 selective solvent via conduit 365. The off-gassed stream of CO2 may be compressed in compressor 367 to a pressure of 1.0 MPa to 6.9 MPa, if necessary, and cooled to a temperature of −50° C. to 40° C. in heat exchanger 369, if necessary, to liquefy the CO2. The liquefied CO2 may be dried and stored in a liquid CO2 storage tank 371, which may be fluidly operatively coupled to the CO2 solvent regenerator 359 via conduit 373.

The CO2-depleted gas stream may be treated to separate CO from H2 and N2. The CO2-depleted gas stream may be provided to a CO absorber 375, where the CO absorber may be fluidly operatively coupled to the CO2 absorber 355 via conduit 374 to receive the CO2-depleted gas stream therefrom. The CO absorber 375 may be a scrubber in which the CO2-depleted gas stream may be contacted with a solvent having a high selectivity for CO uptake relative to H2 or N2 such as an aromatic hydrocarbon solution of cuprous aluminum chloride. Selective CO absorption using an aromatic solution of cuprous aluminum chloride may be known to those of ordinary skill in the art as the COPURESM process. A CO-depleted gas stream and a CO-enriched solvent stream may be produced by contacting the CO2-depleted gas stream with the CO selective solvent in the CO absorber 375, for example at a temperature of from 0° C. to 40° C.

The CO-enriched solvent stream may be treated to separate CO from the solvent. The CO absorber 375 may be fluidly operatively coupled to a CO solvent regenerator 377 to provide the CO-enriched solvent stream to the CO solvent regenerator via conduit 379. The CO selective solvent may be regenerated and a CO off-gas stream may be produced in the CO solvent regenerator 377 by heating the CO enriched solvent stream to a temperature of from 60° C. to 130° C. and optionally decompressing the CO enriched solvent stream to a pressure of less than 1 MPa. The regenerated CO selective solvent may be cooled in a heat exchanger 381 to a temperature of from 0° C. to 40° C. and returned to the CO absorber 375, where the CO absorber may be fluidly operatively coupled to the CO solvent regenerator 377 to receive the regenerated CO selective solvent therefrom via conduit 383. The off-gassed stream of CO may be compressed in a compressor 385 to a pressure of from 6.9 MPa to 13.8 MPa and the compressed CO gas stream may be cooled against cooling water to a temperature of from 10° C. to 65° C. in a heat exchanger 387, where the cooled compressed CO gas stream may be stored in a CO storage facility 389. The CO storage facility 389 may be fluidly operatively coupled to the CO solvent regenerator 377 via conduit 391 to receive the CO gas stream therefrom.

The CO-depleted gas stream may be treated to separate H2 and N2 gases. The CO-depleted gas stream may be provided to an H2 absorber 393, where the H2 absorber may be fluidly operatively coupled to the CO absorber 375 via conduit 395 to receive the CO-depleted gas stream therefrom. The H2 absorber may be a conventional pressure swing absorber configured to separate H2 from other gases. Separation of H2 from the CO-depleted gas stream in the H2 absorber produces an H2 gas stream and an N2 rich gas stream. The N2 rich gas stream may be discharged to a flare stack or may be vented through a turbo expander to produce shaft power. The H2 gas stream may be compressed in a compressor 397 to a pressure of from 6.9 MPa to 13.8 MPa, the compressed H2 gas stream may be cooled against cooling water in a heat exchanger 399 to a temperature of from 10° C. to 65° C., and the cooled compressed H2 gas stream may be stored in an H2 storage facility 401. The H2 storage facility 401 may be fluidly operatively coupled to the H2 absorber 393 via conduit 403 to receive the H2 gas stream therefrom.

H2 and CO may be provided to a methanol reactor 405 for the production of methanol according to the reaction CO+2H2⇄CH3OH. The methanol reactor 405 may be fluidly operatively coupled to the H2 storage facility 401 by conduit 407 to receive H2 from the H2 storage facility, and may be fluidly operatively coupled to the CO storage facility 389 by conduit 409 to receive CO from the CO storage facility. The H2 gas from the H2 storage facility may be provided to the methanol reactor 405 at a molar ratio of 2:1 relative to the CO gas provided to the methanol reactor. The H2 and CO gases may be provided to the methanol reactor at a pressure of from 5 MPa to 10 MPa, where the H2 and CO gases may be expanded in an expander 411 if necessary prior to introduction to the methanol reactor. The H2 and CO gases may be reacted in the methanol reactor 405 to produce methanol at a temperature of from 200° C. to 300° C. over a Cu/ZnO/Al2O3 catalyst as is conventional in the art of methanol synthesis.

Methanol produced in the methanol reactor 405 and H2S from the H2S storage tank 351 may be provided to a DMS reactor 413 for the production of DMS according to the reaction 2CH3OH+H2S→DMS+2H2O. The DMS reactor 413 may be fluidly operatively coupled to the methanol reactor 405 by conduit 415 to receive methanol therefrom, and may be fluidly operatively coupled to the H2S storage tank 351 by conduit 417 to receive H2S therefrom. The methanol from the methanol reactor 405 may be provided to the DMS reactor 413 at a molar ratio of from 2.1:1 to 2.5:1 relative to the H2S provided to the DMS reactor, where a slight molar excess of methanol is preferred to selectively produce DMS rather than methanethiol. The methanol and H2S may be reacted in the DMS reactor 413 to produce DMS at a temperature of from 320° C. to 440° C. over a La2O3/Al2O3, γ-Al2O3, or WO3/ZrO2 catalyst.

DMS may be recovered from the DMS reactor for use in an oil recovery process. The DMS may be incorporated into an oil recovery formulation for introduction into an oil-bearing formation as part of an oil recovery process. The DMS may be utilized as a solvent for bitumen recovery from oil sands by a non-aqueous leaching process. The DMS may be used as an agent to remediate asphaltene deposition in an oil-bearing formation as a tar mat or as a near-wellbore deposit. DMS may also be utilized to remediate asphaltene flocculation in oil wells and pipelines.

Water produced in the DMS reactor 413 may be cooled in heat exchanger 419 and provided to the boiler 303, where the DMS reactor 413 may be fluidly operatively coupled to the boiler 303 by conduit 421 to provide water from the DMS reactor to the boiler.

In operation, excess methanol may be produced relative to the quantity of H2S separated from the gas stream produced by the coker 301 that is required to produce DMS. Excess methanol may be separated from the methanol reactor 405 and utilized in one or more processes to produce further useful products. The excess methanol may be utilized in a conventional methanol to gasoline process to produce gasoline. Alternatively, the excess methanol may be utilized in a conventional methanol-to-olefins process to produce olefins. Alternatively, additional H2S may be imported into the system for reaction with the excess methanol in the DMS reactor 413 to produce further DMS, where the additional H2S may be imported from an H2S source, for example H2S separated from a sour gas.

DMS as an EOR Agent Example 1. The quality of dimethyl sulfide as an oil recovery agent based on the miscibility of dimethyl sulfide with a crude oil relative to other compounds was evaluated. The miscibility of dimethyl sulfide, ethyl acetate, o-xylene, carbon disulfide, chloroform, dichloromethane, tetrahydrofuran, and pentane solvents with Muskeg River mined oil sands was measured by extracting the oil sands with the solvents at 10° C. and at 30° C. to determine the fraction of hydrocarbons extracted from the oil sands by the solvents. The bitumen content of the Muskeg River mined oil sands was measured at 11 wt. % as an average of bitumen extraction yield values for solvents known to effectively extract substantially all of bitumen from oil sands—in particular chloroform, dichloromethane, o-xylene, tetrahydrofuran, and carbon disulfide. One oil sands sample per solvent per extraction temperature was prepared for extraction, where the solvents used for extraction of the oil sands samples were dimethyl sulfide, ethyl acetate, o-xylene, carbon disulfide, chloroform, dichloromethane, tetrahydrofuran, and pentane. Each oil sands sample was weighed and placed in a cellulose extraction thimble that was placed on a porous polyethylene support disk in a jacketed glass cylinder with a drip rate control valve. Each oil sands sample was then extracted with a selected solvent at a selected temperature (10° C. or 30° C.) in a cyclic contact and drain experiment, where the contact time ranged from 15 to 60 minutes. Fresh contacting solvent was applied and the cyclic extraction repeated until the fluid drained from the apparatus became pale brown in color.

The extracted fluids were stripped of solvent using a rotary evaporator and thereafter vacuum dried to remove residual solvent. The recovered bitumen samples all had residual solvent present in the range of from 3 wt. % to 7 wt. %. The residual solids and extraction thimble were air dried, weighed, and then vacuum dried. Essentially no weight loss was observed upon vacuum drying the residual solids, indicating that the solids did not retain either extraction solvent or easily mobilized water. Collectively, the weight of the solid or sample and thimble recovered after extraction plus the quantity of bitumen recovered after extraction divided by the weight of the initial oil sands sample plus the thimble provide the mass closure for the extractions. The calculated percent mass closure of the samples was slightly high because the recovered bitumen values were not corrected for the 3 wt. % to 7 wt. % residual solvent. The extraction experiment results are summarized in Table 1.

TABLE 1
Summary of Extraction Experiments of Bituminous Oil Sands with Various Fluids

| | | Input | Output | | | |
|---|---|---|---|---|---|---|
| | | Solids | Solids | Weight | Recovered | Experimental |
| | Temperature, | weight, | weight, | Change, | Bitumen, | Weight |
| Extraction Fluid | C. | g | g | g | g | Closure, % |
| Carbon Disulfide | 30 | 151.1 | 134.74 | 16.4 | 16.43 | 100.0 |
| Carbon Disulfide | 10 | 151.4 | 134.62 | 16.8 | 16.62 | 99.9 |
| Chloroform | 30 | 153.7 | 134.3 | 19.4 | 18.62 | 99.5 |
| Chloroform | 10 | 156.2 | 137.5 | 18.7 | 17.85 | 99.5 |
| Dichloromethane | 30 | 155.8 | 138.18 | 17.7 | 16.30 | 99.1 |
| Dichloromethane | 10 | 155.2 | 136.33 | 18.9 | 17.66 | 99.2 |
| o-Xylene | 30 | 156.1 | 136.58 | 19.5 | 17.37 | 98.6 |
| o-Xylene | 10 | 154.0 | 136.66 | 17.3 | 17.36 | 100.0 |
| Tetrahydrofuran | 30 | 154.7 | 136.73 | 18.0 | 17.67 | 99.8 |
| Tetrahydrofuran | 10 | 154.7 | 136.98 | 17.7 | 16.72 | 99.4 |
| Ethyl Acetate | 30 | 153.5 | 135.81 | 17.7 | 11.46 | 96.0 |
| Ethyl Acetate | 10 | 155.7 | 144.51 | 11.2 | 10.32 | 99.4 |
| Pentane | 30 | 154.0 | 139.11 | 14.9 | 13.49 | 99.1 |
| Pentane | 10 | 152.7 | 138.65 | 14.1 | 13.03 | 99.3 |
| Dimethyl Sulfide | 30 | 154.2 | 137.52 | 16.7 | 16.29 | 99.7 |
| Dimethyl Sulfide | 10 | 151.7 | 134.77 | 16.9 | 16.55 | 99.7 |

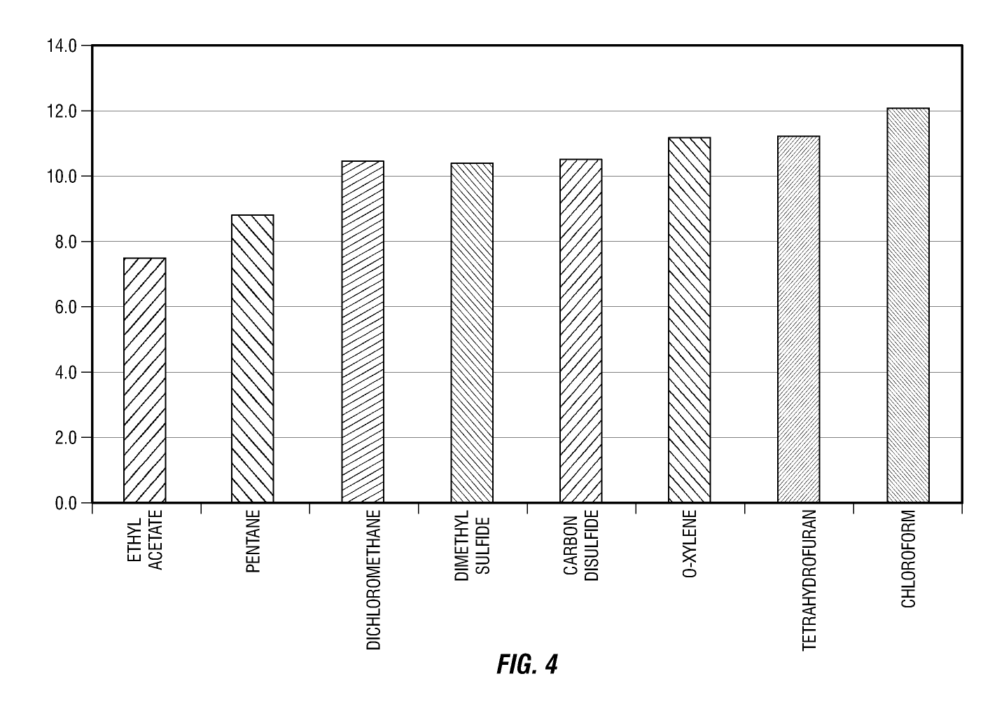

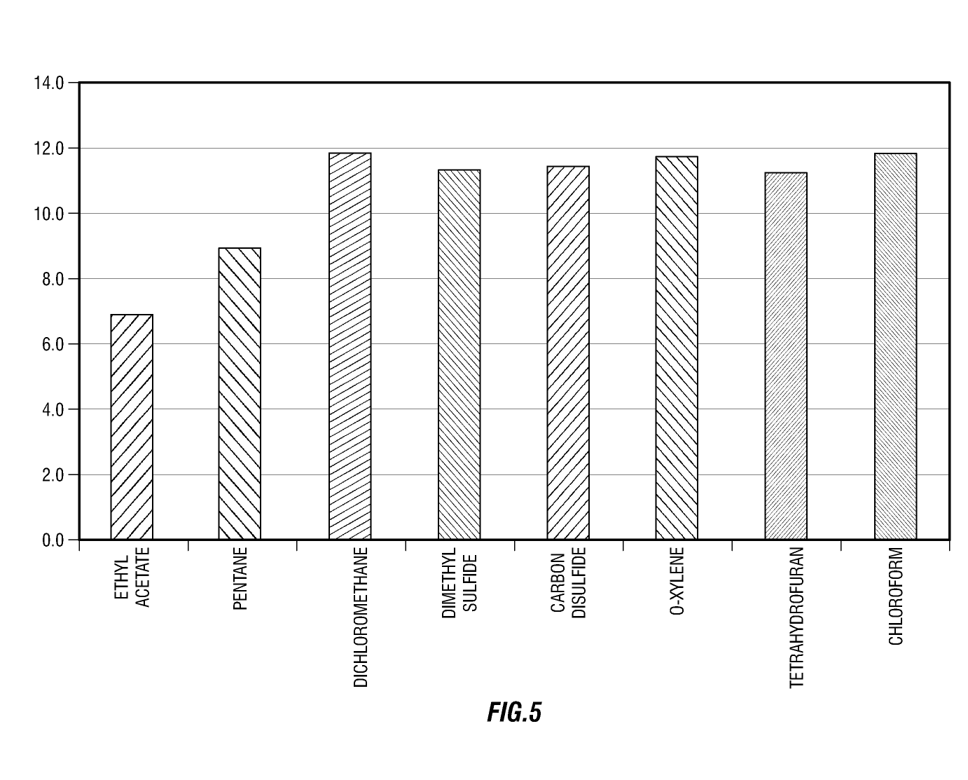

FIG. 4 provides a graph plotting the weight percent yield of extracted bitumen as a function of the extraction fluid at 30° C. applied with a correction factor for residual extraction fluid in the recovered bitumen, and FIG. 5 provides a similar graph for extraction at 10° C. without a correction factor. FIGS. 4 and 5 and Table 1 show that dimethyl sulfide is comparable for recovering bitumen from an oil sand material with the best known fluids for recovering bitumen from an oil sand material—o-xylene, chloroform, carbon disulfide, dichloromethane, and tetrahydrofuran—and is significantly better than pentane and ethyl acetate.

The bitumen samples extracted at 30° C. from each oil sands sample were evaluated by SARA analysis to determine the saturates, aromatics, resins, and asphaltenes composition of the bitumen samples extracted by each solvent. The results are shown in Table 2.

TABLE 2
SARA Analysis of Extracted Bitumen Samples
as a Function of Extraction Fluid

| | Oil Composition Normalized Weight Percent | | | |
|---|---|---|---|---|
| Extraction Fluid | Saturates | Aromatics | Resins | Asphaltenes |
| Ethyl Acetate | 21.30 | 53.72 | 22.92 | 2.05 |
| Pentane | 22.74 | 54.16 | 22.74 | 0.36 |
| Dichloromethane | 15.79 | 44.77 | 24.98 | 14.45 |
| Dimethyl Sulfide | 15.49 | 47.07 | 24.25 | 13.19 |
| Carbon Disulfide | 18.77 | 41.89 | 25.49 | 13.85 |
| o-Xylene | 17.37 | 46.39 | 22.28 | 13.96 |
| Tetrahydrofuran | 16.11 | 45.24 | 24.38 | 14.27 |
| Chloroform | 15.64 | 43.56 | 25.94 | 14.86 |

The SARA analysis showed that pentane and ethyl acetate were much less effective for extraction of asphaltenes from oil sands than are the known highly effective bitumen extraction fluids dichloromethane, carbon disulfide, o-xylene, tetrahydrofuran, and chloroform. The SARA analysis also showed that dimethyl sulfide has excellent miscibility properties for even the most difficult hydrocarbons—asphaltenes.

The data showed that dimethyl sulfide is generally as good as the recognized very good bitumen extraction fluids for recovery of bitumen from oil sands, and is highly compatible with saturates, aromatics, resins, and asphaltenes.

DMS as an EOR Agent Example 2. The quality of dimethyl sulfide as an oil recovery agent based on the crude oil viscosity lowering properties of dimethyl sulfide was evaluated. Three crude oils having widely disparate viscosity characteristics—an African Waxy crude, a Middle Eastern asphaltic crude, and a Canadian asphaltic crude—were blended with dimethyl sulfide. Some properties of the three crudes are provided in Table 3.

TABLE 3
Crude Oil Properties

| | | Middle | |
|---|---|---|---|
| | African | Eastern | Canadian |
| | Waxy | Asphaltic | Asphaltic |
| | crude | crude | Crude |
| Hydrogen (wt. %) | 13.21 | 11.62 | 10.1 |
| Carbon (wt. %) | 86.46 | 86.55 | 82 |
| Oxygen (wt. %) | na | na | 0.62 |
| Nitrogen (wt. %) | 0.166 | 0.184 | 0.37 |
| Sulfur (wt. %) | 0.124 | 1.61 | 6.69 |
| Nickel (ppm wt.) | 32 | 14.2 | 70 |
| Vanadium (ppm wt.) | 1 | 11.2 | 205 |
| microcarbon residue (wt. %) | na | 8.50 | 12.5 |
| C₅ Asphaltenes (wt. %) | <0.1 | na | 16.2 |
| C₇ Asphaltenes (wt. %) | <0.1 | na | 10.9 |
| Density (g/ml) (15.6° C.) | 0.88 | 0.9509 | 1.01 |
| API Gravity (15.6° C.) | 28.1 | 17.3 | 8.5 |
| Water (Karl Fisher Titration) (wt. %) | 1.65 | <0.1 | <0.1 |
| TAN-E (ASTM D664) (mg KOH/g) | 1.34 | 4.5 | 3.91 |
| Volatiles Removed by Topping, wt % | 21.6 | 0 | 0 |
| Saturates in Topped Fluid, wt. % | 60.4 | 41.7 | 12.7 |
| Aromatics in Topped Fluid, wt. % | 31.0 | 40.5 | 57.1 |
| Resin in Topped Fluid, wt. % | 8.5 | 14.5 | 17.1 |
| Asphaltenes in Topped Fluid, wt. % | 0.1 | 3.4 | 13.1 |

Boiling Range Distribution

| | | | |
|---|---|---|---|
| Initial Boiling Point-204° C. (wt. %) | 8.5 | 3.0 | 0 |
| 204° C. (400° F.)-260° C. (wt. %) | 9.5 | 5.8 | 1.0 |
| 260° C. (500° F.)-343° C. (wt. %) | 16.0 | 14.0 | 14.0 |
| 343° C. (650° F.)-538° C. (wt. %) | 39.5 | 42.9 | 38.0 |
| >538° C. (wt. %) | 26.5 | 34.3 | 47.0 |

A control sample of each crude was prepared containing no dimethyl sulfide, and samples of each crude were prepared and blended with dimethyl sulfide to prepare crude samples containing increasing concentrations of dimethyl sulfide. Each sample of each of the crudes was heated to 60° C. to dissolve any waxes therein and to permit weighing of a homogeneous liquid, weighed, allowed to cool overnight, then blended with a selected quantity of dimethyl sulfide. The samples of the crude/dimethyl sulfide blend were then heated to 60° C. and mixed to ensure homogeneous blending of the dimethyl sulfide in the samples. Absolute (dynamic) viscosity measurements of each of the samples were taken using a rheometer and a closed cup sensor assembly. Viscosity measurements of each of the samples of the West African waxy crude and the Middle Eastern asphaltic crude were taken at 20° C., 40° C., 60° C., 80° C., and then again at 20° C. after cooling from 80° C., where the second measurement at 20° C. is taken to measure the viscosity without the presence of waxes since wax formation occurs slowly enough to permit viscosity measurement at 20° C. without the presence of wax. Viscosity measurements of each of the samples of the Canadian asphaltic crude were taken at 5° C., 10° C., 20° C., 40° C., 60° C., 80° C., The measured viscosities for each of the crudes are shown in Tables 4, 5, and 6 below.

TABLE 4
Viscosity (mPa s) of West African Waxy Crude vs. Temperature
at Various levels of Dimethyl Sulfide Diluent

| DMS, wt. % | 20° C. | 40° C. | 60° C. | 80° C. | 20° C. |
|---|---|---|---|---|---|
| 0.00 | 128.8 | 34.94 | 15.84 | 9.59 | 114.4 |
| 1.21 | 125.8 | 30.94 | 14.66 | 8.92 | 100.1 |
| 2.48 | 122.3 | 30.53 | 13.66 | 8.44 | 89.23 |
| 5.03 | 78.37 | 20.24 | 10.45 | 6.55 | 55.21 |
| 7.60 | 60.92 | 17.08 | 9.29 | 6.09 | 40.89 |
| 9.95 | 44.70 | 13.03 | 7.58 | 5.04 | 30.61 |
| 15.13 | 23.96 | 8.32 | 4.97 | 3.38 | 17.64 |
| 19.30 | 15.26 | 6.25 | 4.05 | 2.92 | 12.06 |

TABLE 5
Viscosity (mPa s) of Middle Eastern Asphaltic Crude vs.
Temperature at Various levels of Dimethyl Sulfide Diluent

| DMS, wt. % | 20° C. | 40° C. | 60° C. | 80° C. | 20° C. |
|---|---|---|---|---|---|
| 0.00 | 2936.3 | 502.6 | 143.6 | 56.6 | 2922.7 |
| 1.3 | 1733.8 | 334.5 | 106.7 | 44.6 | 1624.8 |
| 2.6 | 1026.6 | 219.9 | 76.5 | 34.3 | 881.1 |
| 5.3 | 496.5 | 134.2 | 52.2 | 25.5 | 503.5 |
| 7.6 | 288.0 | 89.4 | 37.4 | 19.3 | 290.0 |
| 10.1 | 150.0 | 52.4 | 24.5 | 13.5 | 150.5 |
| 15.2 | 59.4 | 25.2 | 13.6 | 8.2 | 60.7 |
| 20.1 | 29.9 | 14.8 | 8.7 | 5.7 | 31.0 |

TABLE 6
Viscosity (mPa s) of Topped Canadian Asphaltic Crude vs.
Temperature at Various levels of Dimethyl Sulfide Diluent

| DMS, wt. % | 5° C. | 10° C. | 20° C. | 40° C. | 60° C. | 80° C. |
|---|---|---|---|---|---|---|
| 0.00 | | | 579804 | 28340 | 3403 | 732 |
| 1.43 | | | 212525 | 14721 | 2209 | 538 |
| 2.07 | | | 134880 | 10523 | 1747 | 427 |
| 4.87 | | | 28720 | 3235 | 985 | 328 |
| 8.01 | | | 5799 | 982 | 275 | 106 |
| 9.80 | | | 2760 | 571 | 173 | 73 |
| 14.81 | 1794 | 1155 | 548 | 159 | 64 | 32 |
| 19.78 | | | 188 | 69 | 33 | 19 |
| 29.88 | 113 | 81 | 51 | 22 | 13 | 8 |
| 39.61 | 23 | 20 | 14 | 8 | 6 | 4 |

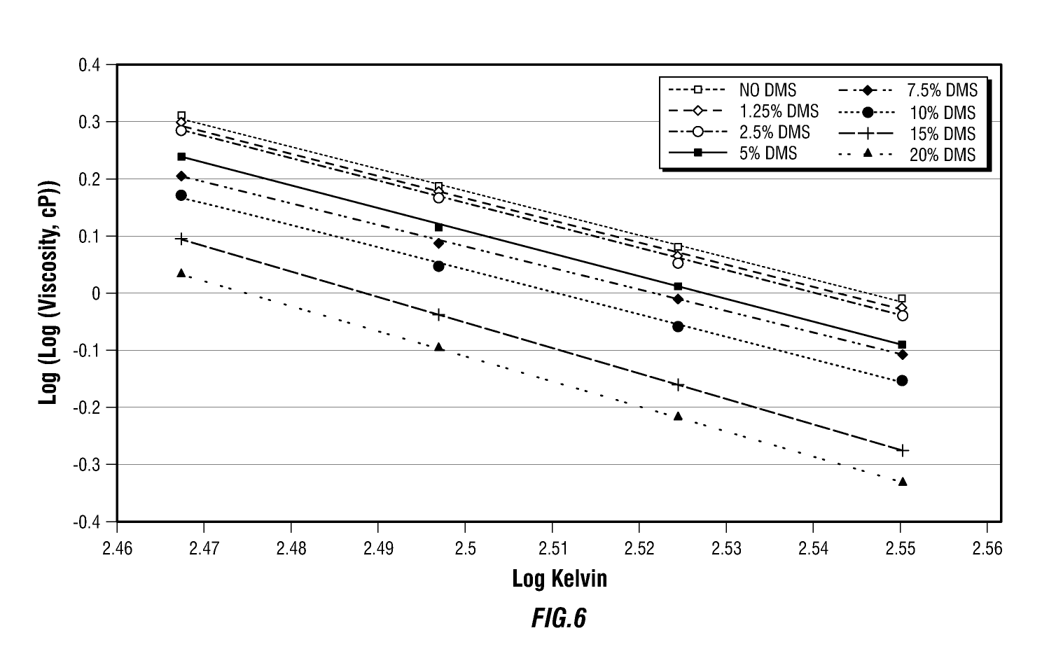

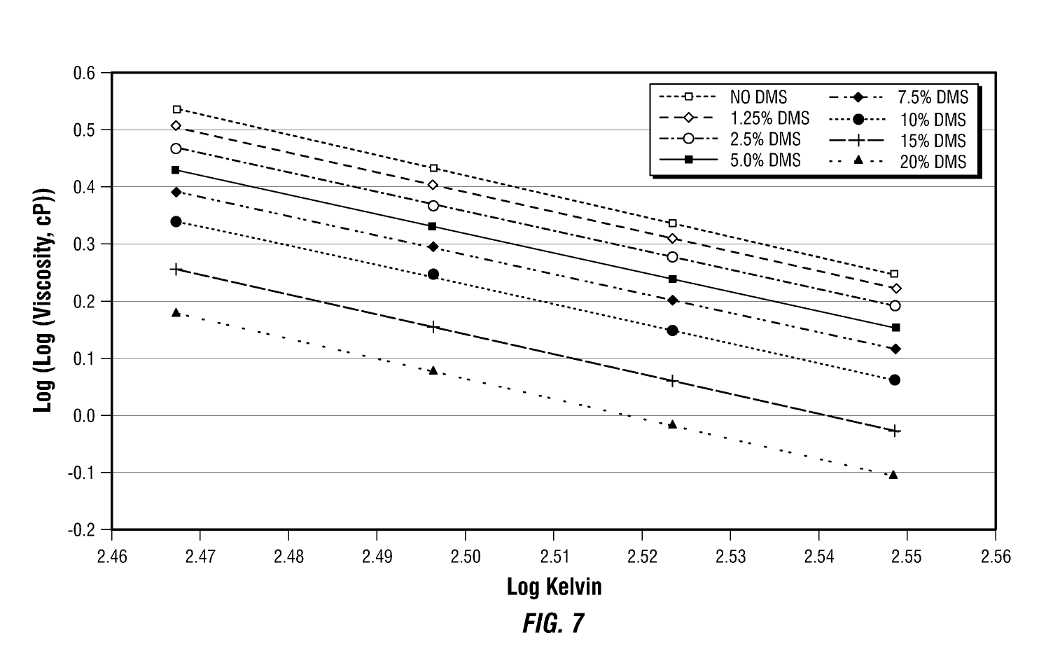

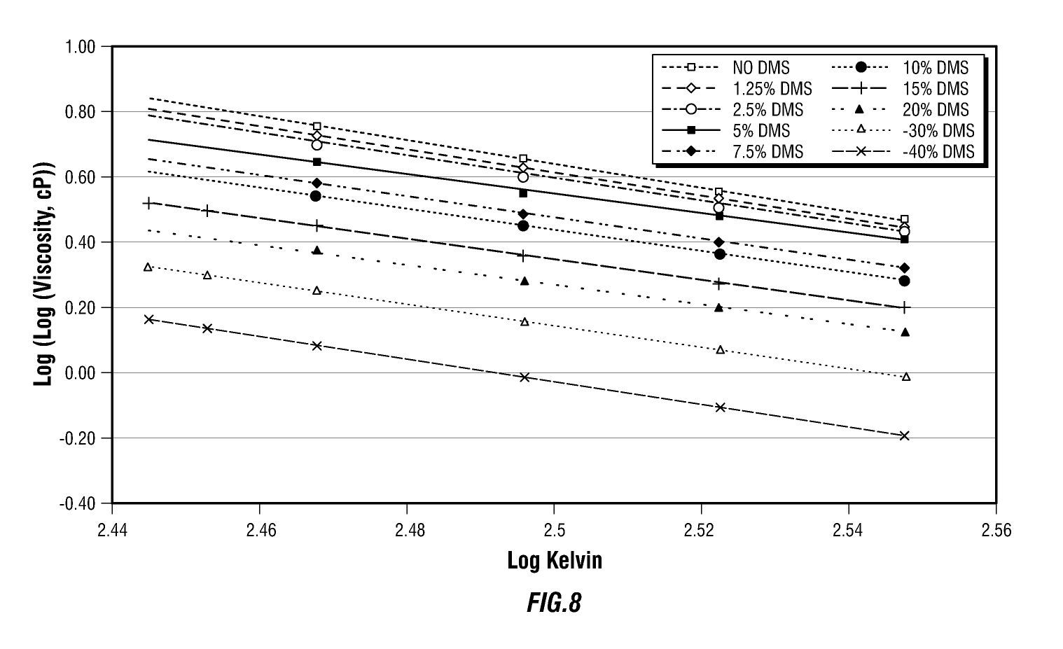

FIGS. 6, 7, and 8 show plots of Log [Log (Viscosity)] v. Log [Temperature °K] derived from the measured viscosities in Tables 4, 5, and 6, respectively, illustrating the effect of increasing concentrations of dimethyl sulfide in lowering the viscosity of the crude samples.

The measured viscosities and the plots show that dimethyl sulfide is effective for significantly lowering the viscosity of a crude oil over a wide range of initial crude oil viscosities.

### DMS as an EOR Agent Example 3

Incremental recovery of oil from a formation core using an oil recovery formulation consisting of dimethyl sulfide following oil recovery from the core by water-flooding was measured to evaluate the effectiveness of DMS as a tertiary oil recovery agent.

Two 5.02 cm long Berea sandstone cores with a core diameter of 3.78 cm and a permeability between 925 and 1325 mD were saturated with a brine having a composition as set forth in Table 7.

TABLE 7
Brine Composition

| Chemical | | | | | | |
|---|---|---|---|---|---|---|
| component | CaCl₂ | MgCl₂ | KCl | NaCl | Na₂SO₄ | NaHCO₃ |
| Concentration | 0.386 | 0.523 | 1.478 | 28.311 | 0.072 | 0.181 |
| (kppm) | | | | | | |

After saturation of the cores with brine, the brine was displaced by a Middle Eastern Asphaltic crude oil having the characteristics as set forth above in Table 3 to saturate the cores with oil.

Oil was recovered from each oil saturated core by the addition of brine to the core under pressure and by subsequent addition of DMS to the core under pressure. Each core was treated as follows to determine the amount of oil recovered from the core by addition of brine followed by addition of DMS. Oil was initially displaced from the core by addition of brine to the core under pressure. A confining pressure of 1 MPa was applied to the core during addition of the brine, and the flow rate of brine to the core was set at 0.05 ml/min. The core was maintained at a temperature of 50° C. during displacement of oil from the core with brine. Oil was produced and collected from the core during the displacement of oil from the core with brine until no further oil production was observed (24 hours). After no further oil was displaced from the core by the brine, oil was displaced from the core by addition of DMS to the core under pressure. DMS was added to the core at a flow rate of 0.05 ml/min for a period of 32 hours for the first core and for a period of 15 hours for the second core. Oil displaced from the core during the addition of DMS to the core was collected separately from the oil displaced by the addition of brine to the core.

The oil samples collected from each core by brine displacement and by DMS displacement were isolated from water by extraction with dichloromethane, and the separated organic layer was dried over sodium sulfate. After evaporation of volatiles from the separated, dried organic layer of each oil sample, the amount of oil displaced by brine addition to a core and the amount of oil displaced by DMS addition to the core were weighed. Volatiles were also evaporated from a sample of the Middle Eastern asphaltic oil to be able to correct for loss of light-end compounds during evaporation. Table 8 shows the amount of oil produced from each core by brine displacement followed by DMS displacement.

TABLE 8

| | | Oil produced | | Oil produced |
|---|---|---|---|---|
| | Oil produced | Brine dis- | Oil produced | DMS dis- |
| | Brine dis- | placement | DMS dis- | placement |
| | placement | (of % oil ini- | placement | (of % oil ini- |
| | (ml) | tially in core) | (ml) | tially in core) |
| Core 1 | 4.9 | 45 | 3.5 | 32 |
| Core 2 | 5.0 | 45 | 3.3 | 30 |

As shown in Table 8, DMS is quite effective for recovering an incremental quantity of oil from a formation core after recovery of oil from the core by waterflooding with a brine solution—recovering approximately 60% of the oil remaining in the core after the waterflood.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

What is claimed:

1. A method comprising:
providing a gasified coke stream comprising carbon monoxide, hydrogen, hydrogen sulfide, carbon dioxide, and nitrogen;
separating the gasified coke stream into a stream enriched in carbon monoxide relative to the gasified coke stream, a stream enriched in hydrogen relative to the gasified coke stream, and a stream enriched in hydrogen sulfide relative to the gasified coke stream;
producing methanol from at least a portion of the separated carbon monoxide enriched stream and at least a portion of the separated hydrogen enriched stream; and
producing dimethyl sulfide from at least a portion of the produced methanol and at least a portion of the separated hydrogen sulfide enriched stream.

2. The method of claim 1 further comprising:
gasifying coke to produce the gasified coke.

3. The method of claim 2, wherein the coke is a petroleum coke.

4. The method of claim 3, wherein the petroleum coke comprises at least 3 wt. % sulfur.

5. The method of claim 2, wherein the coke is a coal coke.

6. The method of claim 5, wherein the coal coke comprises at least 1 wt. % sulfur.

7. The method of claim 1, further comprising separating from the gasified coke stream a stream enriched in carbon dioxide relative to the gasified coke stream, and compressing the carbon dioxide enriched stream.

8. The method of claim 1 further comprising:
producing gasoline from at least a portion of the methanol.

9. The method of claim 1 further comprising:
producing an olefin from at least a portion of the methanol.

10. The method of claim 1 further comprising:
introducing at least a portion of the dimethyl sulfide into an oil-bearing formation in an oil recovery formulation.

11. The method of claim 10, wherein the oil recovery formulation comprises at least 75 mol % dimethyl sulfide.

12. A system comprising:
a separator that receives a gasified coke stream and is structured and arranged to produce a carbon monoxide stream, a hydrogen stream, and a hydrogen sulfide stream from the gasified coke stream;
a methanol reactor fluidly operatively coupled to the separator to receive at least a portion of the carbon monoxide stream and at least a portion of the hydrogen stream from the separator, wherein the methanol reactor is structured and arranged to produce a methanol stream from the carbon monoxide stream and the hydrogen stream; and
a dimethyl sulfide reactor fluidly operatively coupled to the methanol reactor to receive at least a portion of the methanol stream from the methanol reactor and fluidly operatively coupled to the separator to receive at least a portion of the hydrogen sulfide stream from the separator, wherein the dimethyl sulfide reactor is structured and arranged to produce a dimethyl sulfide stream from the methanol stream and the hydrogen sulfide stream.

13. The system of claim 12 further comprising:
a coke gasification reactor that is structured and arranged to produce the gasified coke stream from coke, wherein the separator is fluidly operatively coupled to the coke gasification reactor to receive the gasified coke stream from the coke gasification reactor.

14. The system of claim 12, wherein the separator is further structured and arranged to produce a carbon dioxide stream, and the system further comprises a condenser fluidly operatively coupled to the separator to receive the carbon dioxide stream.

15. The system of claim 12 further comprising:
a methanol-to-gasoline reactor that is structured and arranged to produce a gasoline stream, wherein the methanol-to-gasoline reactor is fluidly operatively coupled to the methanol reactor to receive the methanol stream therefrom.

16. The system of claim 12 further comprising:
a methanol-to-olefin reactor that is structured and arranged to produce an olefin stream, wherein the methanol-to-olefin reactor is fluidly operatively coupled to the methanol reactor to receive the methanol stream therefrom.
